(19) United States
(12) Patent Application Publication
(10) Pub. No.: US 2010/0217013 A1
(43) Pub. Date: Aug. 26, 2010

(54) ENANTIOSELECTIVE PROCESS FOR THE PREPARATION OF ZOLMITRIPTAN

(76) Inventors: Wenge Li, Plainsboro, NJ (US); Yongxiang Chi, Plainsboro, NJ (US); Jingyang Zhu, Monmouth Junction, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/638,488

(22) Filed: Dec. 15, 2009

Related U.S. Application Data
(60) Provisional application No. 61/122,626, filed on Dec. 15, 2008.

(51) Int. Cl. C07D 209/14

(52) U.S. Cl. 548/504

(57) ABSTRACT

An enantioselective process for preparing zolmitriptan, (S)-4-{[3-[2-(dimethylamine)ethyl]-1H-indol-5-yl]methyl}-2-oxazolidinone), by asymmetric hydrogenation of (Z)-2-(acetylamino)-3-{[3-N,N-(dimethylamine)ethyl)-1H-indol-5-yl]-2-propenoic acid methyl ester in the presence of hydrogen and an enantioselective chiral phosphine transition metal catalyst.

This application claims the benefit under 35 U.S.C. 119(e) of U.S. Provisional Patent Application No. 61/122,626 filed on Dec. 15, 2008.

### BACKGROUND OF THE INVENTION

The present invention relates to a process for preparation of an optical pure indole derivative of (S)-4-{[3-[2-(dimethylamine)ethyl]-1H-indol-5-yl]methyl}-2-oxazolidinone (Formula I), also known as Zolmitriptan.

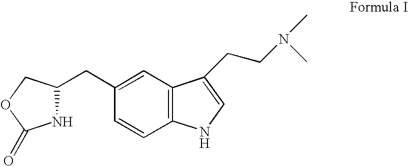

Zolmitriptan has previously been prepared by methodologies that employed substituted phenylalanine as the starting material. Substituted phenylalanine respectively acted as the supplier of the chiral centers in the Zolmitriptan molecule. International Patent Publications of WO 91/18897, WO 97/06162, WO 01/34561, WO 2004/014901, WO 2005/105792 and WO 2008/018090, which are herein incorporated by reference, used phenylalanine and/or its derivatives, such as amino or nitro phenylalanine, as the starting material.

The prior art processes carried the chiral molecule through the entire synthetic route. In most cases, the oxazolidinone ring was prepared during the earlier stages of the processes while the other down stream chemistry, including a Fischer reaction, were conducted under harsh conditions.

The prior art process suffer from multiple disadvantages, among which (1) undesirable racemerization and (2) opening up of the oxazolidinone ring, are worth mentioning.

### SUMMARY OF THE INVENTION

The shortcomings of the prior art are now addressed in the present invention. The object of this invention is to provide an alternative process for the preparation of Zolmitriptan using an asymmetric hydrogenation as the key step. The present invention allows the Fischer reaction to occur at an earlier stage of the synthetic process.

One aspect of the present invention provides an enantioselective process for producing a substituted S-alanine compound having the structure:

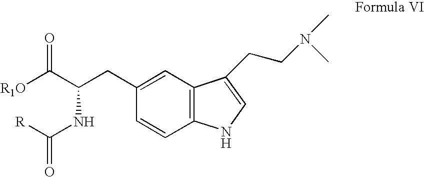

wherein R and R₁ are independently hydrogen or an alkyl group, and the process includes the step of asymmetrically hydrogenating a compound having the structure:

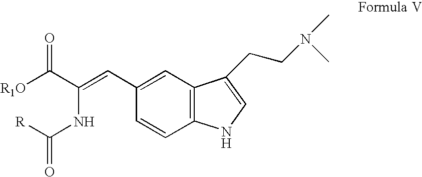

in the presence of hydrogen and an S-directing chiral phosphine transition metal catalyst.

In one embodiment of this aspect of the present invention, the novel process for producing a substituted S-alanine compound employs enantioselective S-directing P-chiral catalysts. Suitable catalysts include S-directing phosphine compounds represented by the Formula M(L)(P*)X, wherein M represents Rh, Ru or iridium; L represents 1,5-cyclooctandiene or 2,5-norbornadiene; P* represents an S-directing chiral monodentate or bidentate phosphine compound, and X represents a tetrahaloborate. In another embodiment of this aspect of the invention, P* is an S-directing enantiomer of a monodentate or bidentate phosphine compound selected from ScRp-DuanPhos, RcSp-DuanPhos, SSRR-TangPhos, BINAP, DuPhos and BPE.

The substituted S-alanine compounds of Formula V are prepared by reacting the compound of Formula IV in which X is a halogen, with methyl 2-acetamido acrylate via a Heck Reaction:

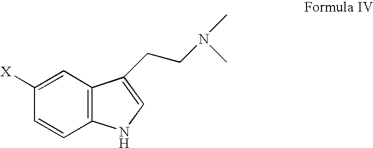

Compounds having the structure of Formula IV are prepared by reacting a compound having the structure of Formula III with 4-N, N-dimethylbutanal diacetal via a Fisher reaction.

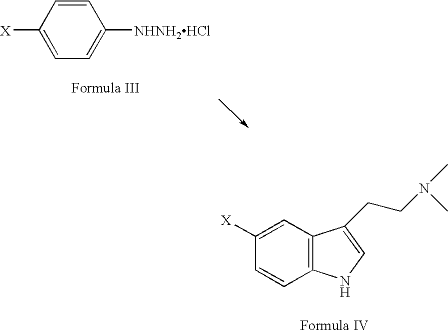

Compounds of Formula III are prepared by reacting the corresponding 4-halo-aniline compound with sodium nitrite.

The S-alanine compounds of Formula VI are reduced to the S-alcohols of Formula VII via a hydride reduction reaction:

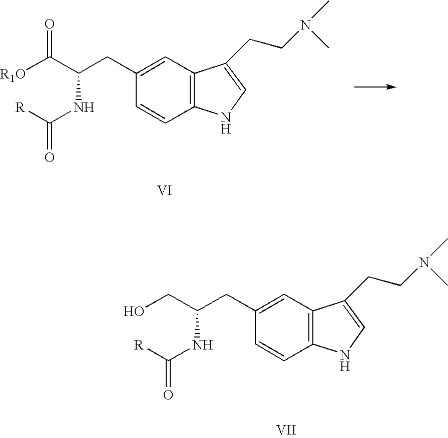

wherein R₁ and R are same as described above.

The Formula VII S-alcohols are converted to an S-amino alcohol hydrochloride of Formula VIII by a hydrolysis reaction:

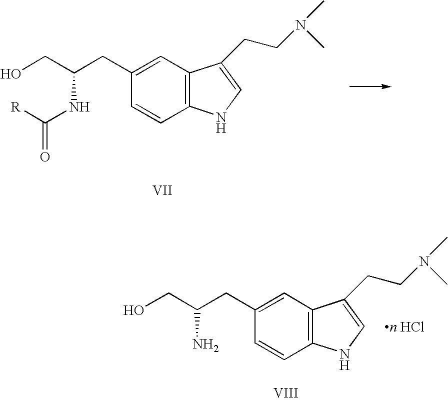

wherein R is same as described above and n=0, 1, 2.

The oxzalidinone ring compound of Formula I is then formed by reacting the compound of Formula VIII with a ring closure agent.

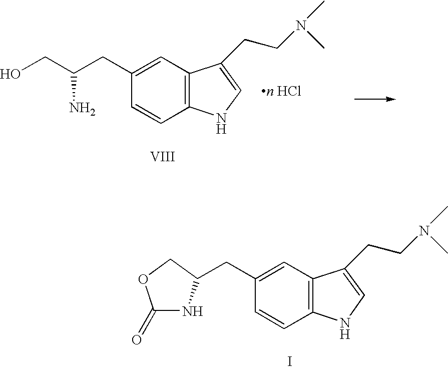

The present invention includes embodiments in which the ring closure reagent is selected from phosgene, diphosgene, triphosgene, ethyl carbonate and 1,1′-carbonyldiimidazole.

In another aspect of the present invention, new intermediate compounds are disclosed that are useful in the synthesis of zolmitriptan are disclosed having the structures of Formulae (V), (VI) and (VII) are disclosed. Accordingly, Formula (V) is

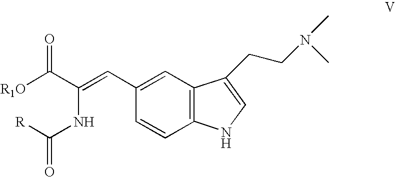

wherein R and R₁ are independently an alkyl group;

Formula (VI) is

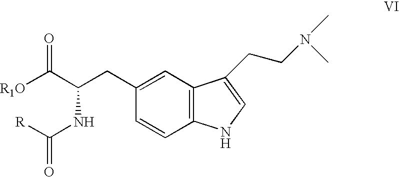

wherein R and R₁ are the same as described above;

and Formula (VII) is

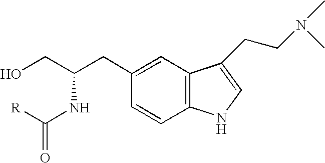

wherein R is the same as described above.

The chiral phosphine transition metal catalysts can be either S-directing or R-directing. Substituting an R-directing catalyst for the S-directing catalyst in the method for producing the S-alanine compound of the present invention provides an R-alanine compound instead.

Accordingly, another aspect of the present invention provides an enantioselective process for producing a substituted R-alanine compound having the structure:

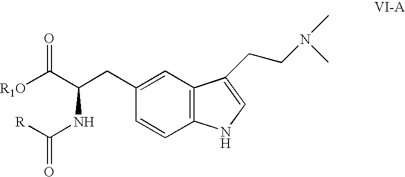

wherein R and R₁ are independently hydrogen or an alkyl group, and the process includes the step of asymmetrically hydrogenating a compound having the structure:

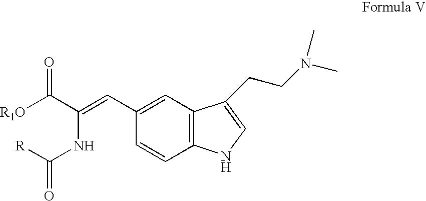

in the presence of hydrogen and an R-directing chiral phosphine transition metal catalyst.

Another aspect of the present invention provides the R-isomer of the compound of Formula (VI) having the structure of Formula (VI-A):

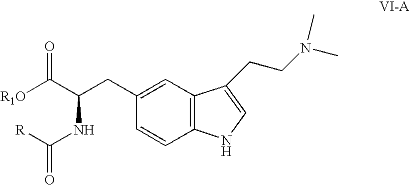

wherein R and R₁ are the same as described above.

### DETAILED DESCRIPTION OF THE PREFERRED EMBODIMENTS

As employed above and throughout the disclosure, the following terms, unless otherwise indicated, shall be understood to have the following meanings:

The term “Alkyl” refers to the radical of saturated aliphatic groups, including straight-chain alkyl groups and branched-chain alkyl groups. The term “cycloalkyl” refers to a non-aromatic mono or multicyclic ring system of about 3 to 7 carbon atoms. Examples of cycloalkyl groups include cyclopropyl, cyclobutyl, cyclopentyl, cyclohexyl and the like.

The term “alkoxy” as used herein refers to an alkyl group, as defined above, having an oxygen radical attached thereto. Representative alkoxyl groups include methyloxy, ethoxy, propoxy, t-Butoxy, benzyloxyl and the like.

The term “halogen” refers to chlorine, bromine, fluorine or iodine. In more preferred embodiments, the halogen is selected from the group consisting of chlorine, bromine and iodine.

The term “chiral phosphine transition metal catalyst” refers to enantioselective catalyst compounds such as those described in U.S. Pat. No. 7,105,702, the teachings of which are incorporated herein by its entirety. The preferred phosphine compounds have the formula M(L)(P*)X , wherein M represents Rh, Ru or iridium; L represents 1,5-cyclooctandiene or 2,5-norbornadiene; P* represents either an S-directing enantiomer or an R-directing enantiomer of a chiral monodentate or bidentate phosphine compound; and X represents tetrafluoroborate and similar anions. Examples of S-directing phosphine compound enantiomers include ScRp-DuanPhos, RcSp-DuanPhos, SSRR-TangPhos, BINAP, DuPhos and BPE. The R-directing counterparts of these phosphine compound are readily identified by those of ordinary skill in the art without undue experimentation.

The term Rh(L)(P*)X type catalysts refers to such catalysts wherein L represents 1,5-cyclooctandiene or 2,5-norbornadiene; P* represents chiral phosphine compounds such as S-directing phosphine compound enantiomers selected from ScRp-DuanPhos, RcSp-DuanPhos, SSRR-TangPhos, BINAP, DuPhos and BPE; and X represents tetrafluoroborate and similar anions.

The enantioselective catalysts can be either S-directing or R-directing. For example, the enantioselective process of the present invention for producing a substituted S-alanine compound can be performed using Rh-(S,S,R,R)-TangPhos as the chiral phosphine transition metal catalyst, but when Rh-(R,R,S,S)-TangPhos is used instead, the R-alanine isomer is obtained. The selection of S-directing vs. R-directing chiral phosphine transition metal catalysts to obtain the desired enantiomer is readily performed by those of ordinary skill in this art without undue experimentation.

The present invention provides a new process for preparing Zolmitriptan which represents an improvement over the prior art and is elucidated in Scheme 1:

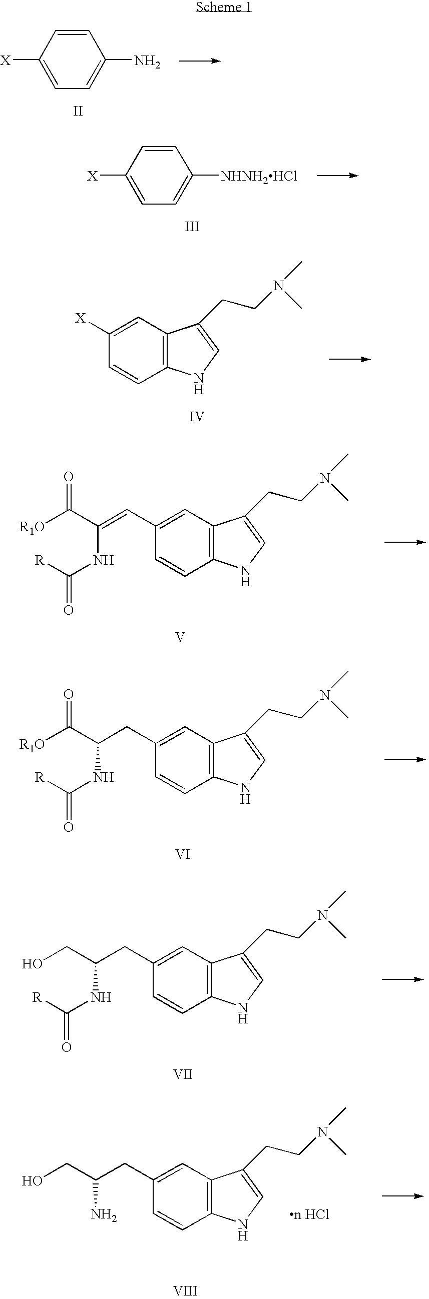

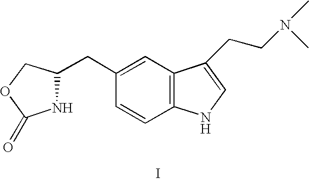

In particular, Zolmitriptan is prepared in a process including the following steps:

(1) Formation of 4-halogen-phenylhydrazine hydrochloride (Formula III) by diazotizing of a 4-halo-aniline, followed by reduction with tin (II) chloride in hydrogen chloride solution wherein X represents a halogen. In a preferred embodiment of the present invention, the halogen is selected from chlorine, bromine and iodine.

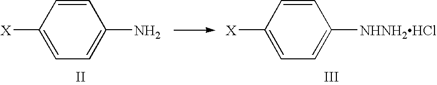

(2) Indole ring closure via the Fischer reaction. Halogen indole derivative (Formula IV) is formed by reaction of the hydrazine hydrochloride of Formula III with 4-N,N-dimethyl butanal diethylacetal under acidic condition at elevated temperature, wherein X represents a halogen. In one embodiment of the present invention, the halogen is selected from of chlorine, bromine and iodine.

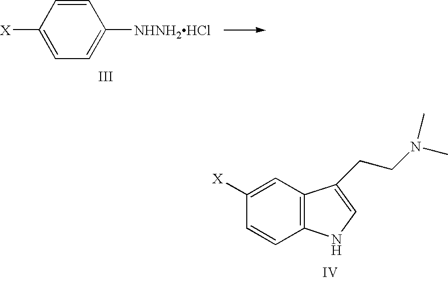

(3) Preparation of substituted acrylate (Formula V) via Heck type reaction.

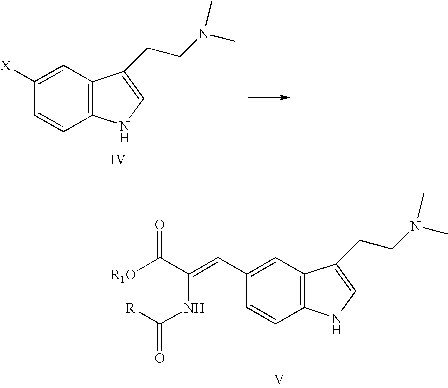

In one embodiment, compound of Formula V is prepared via a Heck type reaction of halogen indole compound of Formula IV with corresponding acrylate, wherein R1 represents alkyl, cycloalkyl and aryl groups, while R represents alkyl, aryl and alkoxy.

In another embodiment of the present invention, the transformation from the compound of Formula IV to the compound of Formula V is achieved by Pd(OAc)₂ catalysis without relying on the presence or absence of phosphine ligands or other additives. In a specific embodiment, this reaction is carried out by employing solvents having a high boiling point, such as N,N-dimethylformamide (DMF), N,N-dimethylacetamide (DMA), N-methyl-2-pyrrolidinone (NMP), dimethyl sulfoxide. In a more specific embodiment the solvent is selected from the group consisting of DMF, DMA and NMP. In a preferred embodiment, NMP is the choice of the solvent.

The reaction temperature of this process step for converting compounds of Formula IV to compounds of Formula V can range from about 50 to about 150° C., preferably in the range of about 100 to about 150° C., and more preferably at the temperature of from about 110 to about 125° C.

Processes according to the present invention use additives such as lithium chloride, tetrabutylammonium chloride, triethyl amine, diisopropylethyl amine, potassium carbonate alone or in combination.

(4) Enantioselective preparation of substituted alanine. Compound VI is prepared via asymmetric hydrogenation of prochiral substrate V.

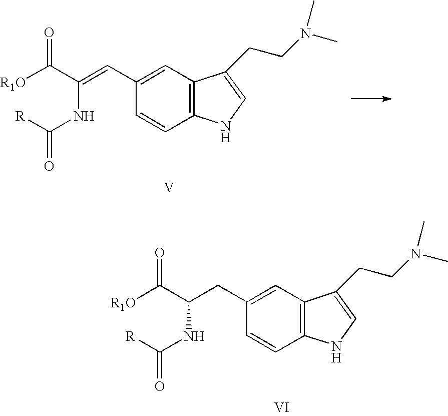

The process of converting the compound V to VI is achieved by using a chiral phosphine transition metal catalyst in the presence of hydrogen, such as the enantioselective P-chiral catalysts described in U.S. Pat. No. 7,105,702, the teachings of which are incorporated herein by its entirety. The preferred phosphine compounds have the formula M(L)(P*)X, wherein M represents Rh, Ru or iridium; L represents 1,5-cyclooctandiene or 2,5-norbornadiene; P* represents an S-directing enantiomer of a monodentate or bidentate chiral phosphine compound such as ScRp-DuanPhos, RcSp-DuanPhos, SSRR-TangPhos, BINAP, DuPhos and BPE; and X represents tetrafluoroborate and similar anions.

In at least one embodiment of this step, the hydrogen pressure can range from about 1 to about 50 bar, preferably in the ranges of about 1 bar to about 30 bar, and most preferably in the range of about 5 to about 10 bar.

The reaction media for this transformation is selected from dichloromethane, methanol, tetrahydrofuran, toluene, ethyl acetate and combinations thereof. In particular, dichloromethane, methanol and tetrahydrofuran are more suitable solvents while methanol is the most suitable solvent. The suitable reaction temperature for converting compounds of Formula V to compounds of Formula VI can range from about 10 to about 50° C., and preferably in range of about 20 to about 30° C.

(5) Formation of the alcohol via hydride reduction.

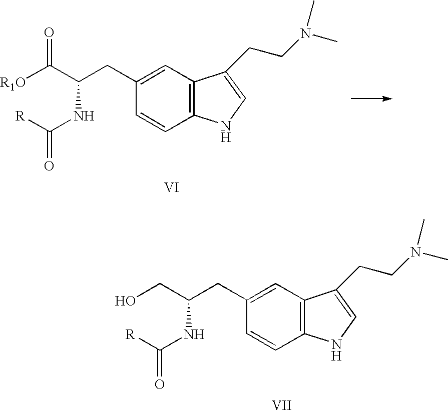

This step of the reaction may be carried out in a media selected from tetrahydrofuran, water, methanol, ethanol, toluene and dichloromethane, or a mixture of two or more of them.

In at least one embodiment of the present invention a suitable hydride reductant is employed selected from sodium borohydride, lithium borohydride and super hydride solution (lithium triethylborohydride solution).

In another aspect of the invention, the step of converting the compound of Formula VI to the compound of Formula VII is carried out at a temperature ranging from about 0 to about 100° C., preferably in the range of about 0 to about 50° C. In a more preferred embodiment, the temperature is about 0 to about 5° C.

(6) Preparation of the amino alcohol hydrochloride by hydrolysis.

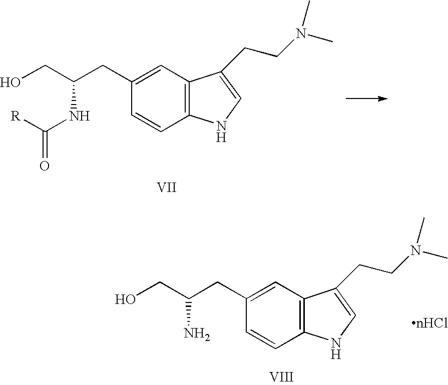

In at least one embodiment of the present invention, the transformation of compounds of Formula VII to compounds of Formula VIII is carried out in a diluted hydrogen chloride solution in water at an elevated temperature. In at least one embodiment, the concentration of hydrogen chloride can be from about 1 to about 10 N. In a more preferred embodiment, the concentration of hydrogen chloride is at about 4 to about 8 N and most preferably at 6 N.

In at least another aspect of the present invention, the step of preparing the compound of Formula VIII is carried out at reaction temperatures ranging from about 50 to about 100° C., preferably from about 80 to about 100° C., and most preferably at a temperature of 95 to 100° C. In the formula for compound VIII, n represents 0, 1, 2, or 3. In a more preferred embodiment, n is 1 or 2 and in the most preferred embodiment, n is 2.

(7) Formation of the oxazolidinone ring.

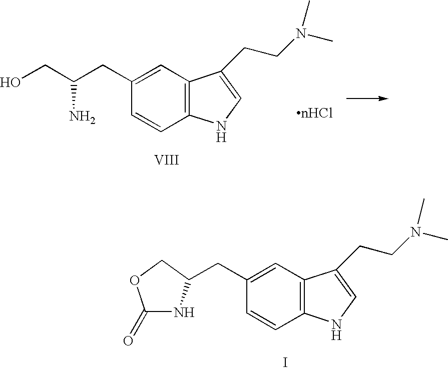

The step of converting the compounds of Formula VIII to the compounds of Formula I is achieved by using ring closure reagents such as phosgene, diphosgene, triphosgene, ethyl carbonate, or 1,1′-carbonyldiimidazole. In particular, the preferred reagents are triphosgene and ethyl carbonate, and in the most preferred embodiment, the reagent is triphosgene.

In one embodiment of the present invention, the step of converting compounds of Formula VIII to the compounds of Formula I is carried out in a reaction media selected from the group consisting of is dichloromethane, toluene, tetrahydrofuran and water, or a mixture of two or more of them. In a more specific embodiment, the reaction media is a mixture of dichloromethane and water.

The invention is further demonstrated in the following examples. The examples are for purposes of illustration only and are not intended to limit the scope of the present invention.

### EXAMPLES

### Example 1

### Preparation of 4-bromophenylhydrazine hydrochloride (III)

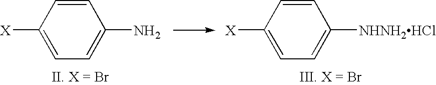

A suspension of 35.34 g of 4-bromoaniline in 200 mL of water and 400 mL of concentrated hydrochloride was cooled to 0° C. To this suspension was added 14.1 g sodium nitrite in 130 mL of water over 30 min. The temperature was maintained at 0 to 5° C. and stirred for another 30 min after addition. The solution was then added at 0° C. over 30 min to a stirred solution of tin (II) chloride (192 g) in 350 mL of concentrated hydrochloride, followed by 3 hours stirring at room temperature. The system was cooled to 5° C. and the solid was collected by filtration and dried at 40° C. under high vacuum to provide 36 g of the product as a white solid.

### ¹H NMR (DMSO-d₆, δ): 6.90 (d, 2H, Ar), 7.44 (d, 2H, Ar), 8.40 (br, 1H), 10.19 (br, 3H).

### Example 2

### Preparation of 2-(5-bromo-1H-indol-3-yl)-N,N-dimethylethanamine (IV)

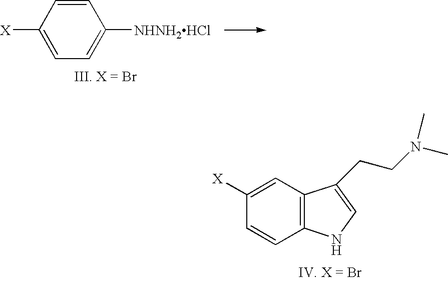

8.3 g of 4-dimethylaminobutanal diethylacetal (Tech grade from Aldrich) was added to a solution of 11.3 g of the product from Example 1 in a mixture of acetic acid (110 mL) and water (5 mL) and the resulting mixture was refluxed for 4 h. The mixture was cooled and evaporated in vacuum. The residue was dissolved in 100 mL of water and the pH was adjusted to 8˜9 by saturated sodium bicarbonate, then extracted with 5×50 mL of dichloromethane. The combined organics were concentrated in vacuo and the residue was eluted through a silica column using DCM/EtOH/NH₄OH (30:8:1) as eluant to give 3.0 g of the desired product as a pale yellow oil.

### ¹H NMR (DMSO-d₆, δ): 2.53 (s, 6H, NMe₂), 2.93 (s, 4H, CH₂CH₂), 7.16 (d, 1H, Ar), 7.26 (s, 1H, Ar), 7.33 (d, 1H, Ar), 7.75 (s, 1H, Ar), 11.20 (s, 1H, NH).

### Example 3

### Preparation of (Z)-2-(acetylamino)-3-{[3-N,N-(dimethylamine)ethyl]-1H-indol-5-yl}-2-propenoic acid methyl ester (V)

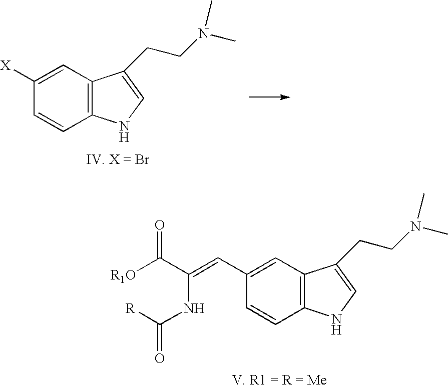

A 100 mL Schleck flask was filled with 2.1 g of the product from Example 2, methyl 2-acetamido acrylate (1.8 g), diisopropylethylamine (4 mL) (o-MePh)₃P (940 mg), Pd(OAc)₂ (172 mg) and NMP (30 mL). Nitrogen atmosphere was applied, a stirring bar was added and the mixture was heated and stirred at 125° C. for 4 h. The mixture was cooled and solvent was removed under high vacuum as much as possible and poured into 50 mL of water. The mixture was then extracted with 5×50 mL of dichloromethane. The combined organics were concentrated in vacuo and the residue was eluted through a silica column using DCM/EtOH/NH4OH (50:8:1) as eluant to give 700 mg of the desired product as a pale yellow oil.

### ¹H NMR (CD₃OD, δ): 2.16 (s, 3H, CH₃CO), 2.38 (s, 6H, NMe₂), 2.68-2.74 (m, 2H), 2.92-3.00 (m, 2H), 3.81 (s, 3H, OMe), 7.12 (s, 1H), 7.35 (d, 1H), 7.42 (d, 1H), 7.64 (s, 1H), 7.87 (s, 1H).

### Example 4

### Preparation of (S)-2-(acetylamino)-3-{[3-N,N-(dimethylamine)ethyl]-1H-indol-5-yl}-propanoic acid methyl ester (VI)

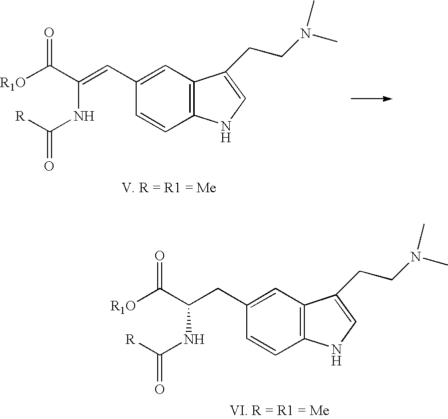

In a glove box, 560 mg of the product from Example 3, 2.3 mg of Rh(COD)[(−)-DuanPhos]BF₄ and 5 mL of air-free methanol was charged to a 20-mL vial. A magnetic stirring bar was also added and the vial was inserted into a 300 mL Parr reactor. Then the reactor was taken out of the glove box. The reactor was charged with 20 bar hydrogen and stirred at room temperature for 24 h. After the hydrogen was released carefully, the reaction mixture was concentrated and the residue was passed through a silica column eluted with DCM/EtOH/NH₄OH (50:8:1) to provide 510 mg of the desired product with 98.0% ee. Ee determination method: HPLC Agilent 1100; Chiralpak AD column; Detected at 230 nm; mobile phase acetonitrile/diethylamine=1000:1; flow rate, 0.5 mL/min. Retention time for R-enantiomer was 12.5 min; retention time for S-entiomer was 14.0 min.
¹H NMR (CD₃OD, δ): 1.90 (s, 3H, CH₃CO), 2.44 (s, 6H, NMe₂), 2.75-2.80 (m, 2H), 2.94-2.99 (m, 2H), 3.00-3.04 (m, 1H), 3.19-3.25 (m, 1H), 3.67 (s, 3H, OMe), 6.95 (d, 1H), 7.07 (s, 1H), 7.26 (d, 1H), 7.37 (s, 1H).

### Example 5

### Preparation of (S)-2-(acetylamino)-3-{[3-N,N-(dimethylamine)ethyl]-1H-indol-5-yl}-1-propanol (VII)

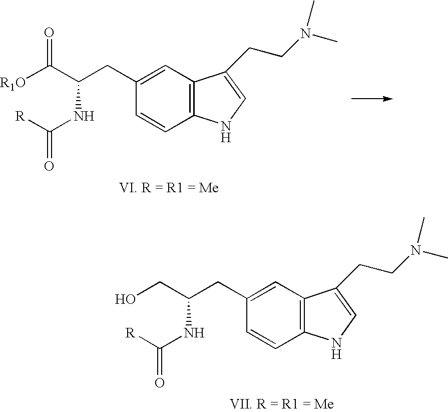

Super hydride® solution (1.0 M in THF, 2.2 mL) was added to a stirred solution of the product from Example 4 (331 mg) in 10 mL of tetrahydrofuran at 0° C. The reaction mixture was stirred at 0° C. for 1 h. The reaction was quenched with water (5 mL) and the aqueous layer was extracted with 5×20 mL of dichloromethane. The combined organics were dried over Na₂SO₄ and concentrated. The residue was purified by silica chromatography eluting with DCM/EtOH/NH₄OH (30:8:1) to provide 170 mg of the desired product.
¹H NMR (CD₃OD, δ): 1.89 (s, 3H, CH₃CO), 2.34 (s, 6H, NMe₂), 2.61-2.67 (m, 2H), 2.76-2.83 (m, 1H), 2.89-3.00 (m, 3H), 3.48-3.62 (m, 2H), 4.08-4.20 (m, 1H), 6.98 (d, 1H), 7.00 (s, 1H), 7.24 (d, 1H), 7.38 (s, 1H).

### Example 6

### Preparation of (S)-2-amino-3-{[3-N,N-(dimethyl-amine)ethyl]-1H-indol-5-yl}-1-propanol dihydrochloride (VIII)

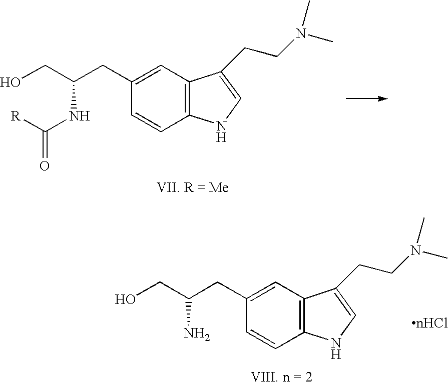

170 mg of the product from Example 5 was dissolved in 9 mL of 4N hydrochloric acid and the mixture was refluxed for 5 h under nitrogen. After cooling the mixture to room temperature, the solvent was removed in vacuo and the residue was dried under high vacuum to provide 164 mg of the desired product.

### ¹H NMR (D₂O, δ): 2.70 (s, 6H, NMe₂), 2.83-2.96 (m, 2H), 3.00-3.10 (m, 2H), 3.20-3.25 (m, 2H), 3.45-3.55 (m, 2H), 3.65-3.70 (m, 1H), 6.97 (d, 1H), 7.15 (s, 1H), 7.34 (d, 1H), 7.39 (s, 1H).

### Example 7

### Preparation of (S)-4-{[3-[2-(dimethyl-amine) ethyl]-1H-indol-5-yl]methyl}-2-oxazolidinone (I)

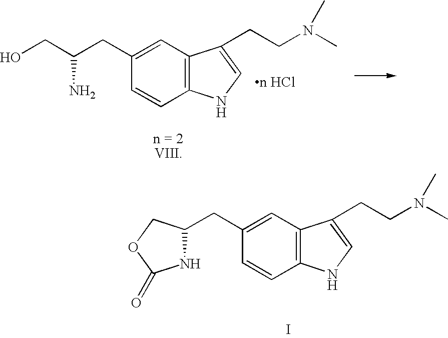

A solution of 164 mg of the product from Example 6 in a mixture of 10 mL water and 10 mL of dichloromethane was cooled to −15° C. and the pH was adjusted to 11 by 5 N aqueous NaOH. A solution of 60 mg of triphosgene in 5 mL of dichloromethane was added at below −10° C. The pH was maintained at 9-11 by periodic additions of 5 N aqueous NaOH. The resulting mixture was stirred at room temperature for 1 h, then the two phases were separated. The aqueous phase was extracted with 5×20 mL of dichloromethane. The combined organics were dried over Na₂SO₄ and concentrated. The residue was purified by silica chromatography eluting with DCM/EtOH/NH₄OH (30:8:1) to provide 95 mg of the desired product.
¹H NMR (DMSO-d₆, δ): 2.2 (s, 6H, NMe₂), 2.5 (m, 2H, CH₂Ar), 2.74-2.94 (m, 4H, CH₂CH₂), 4.0 (m, 2H, CH₂O), 4.2 (m, 1H, CH), 6.9 (d, 1H, Ar), 7.1 (s, 1H, Ar), 7.2 (d, 1H, Ar), 7.3 (s, 1H), 7.7 (s, 1H, NHCO), 10.7 (s, 1H, NH).
¹H NMR (CD₃OD, δ): 2.44 (s, 6H, NMe₂), 2.77-2.81 (m, 2H, CH₂Ar), 2.91-3.0 (m, 4H, CH₂CH₂), 4.17-4.23 (m, 2H, CH₂O), 4.34-4.41 (m, 1H, CH), 6.97 (d, 1H, Ar), 7.07 (s, 1H, Ar), 7.29 (d, 1H, Ar), 7.41 (s, 1H).

While the invention has been disclosed in connection with the preferred embodiments and methods of use, it is to be understood that many alternatives, modifications, and variations thereof are possible without departing from the present invention. Thus, the present invention is intended to embrace all such alternatives, modifications, and variations as may be apparent to those skilled in the art and encompassed within the hereinafter appended claims.

What is claimed:

1. An enantioselective process for producing a substituted (S)-alanine compound having the structure: 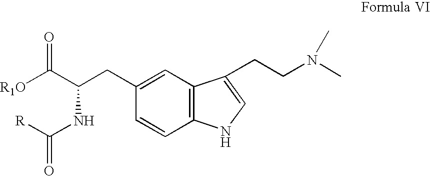
wherein R and R₁ are independently hydrogen or an alkyl group, said process comprising asymetrically hydrogenating a compound having the structure: 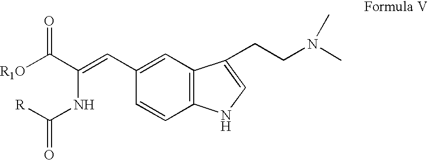
in the presence of hydrogen and an (S)-directing chiral phosphine transition metal catalyst.

2. The process of claim 1, wherein said (S)-directing chiral phosphine transition metal catalyst is an (S)-directing enantioselective P-chiral catalyst represented by the Formula M(L)(P*)X, wherein M represents Rh, Ru or iridium; L represents 1,5-cyclooctandiene or 2,5-norbornadiene; P* represents an (S)-directing chiral monodentate or bidentate phosphine compound, and X represents a tetrahaloborate.

3. The process of claim 2, wherein P* is an (S)-directing enantiomer of a chiral monodentate or bidentate phosphine compound selected from the group consisting of ScRp-DuanPhos, RcSp-DuanPhos, SSRR-TangPhos, BINAP, DuPhos and BPE.

4. The process of claim 1, wherein X is tetrafluoroborate.

5. The enantioselective process of claim 4, further comprising the step of forming an (S)-alcohol via a hydride reduction reaction: 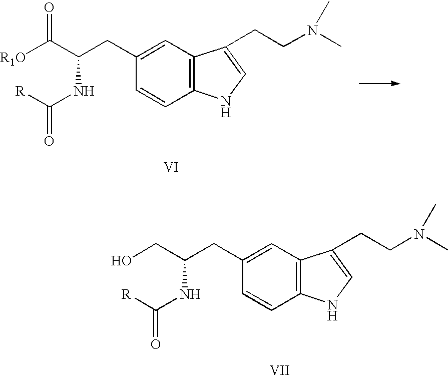
wherein R₁ and R are same as described above.

6. The enantioselective process of claim 5, further comprising the step of forming an (S)-amino alcohol hydrochloride by a hydrolysis reaction: 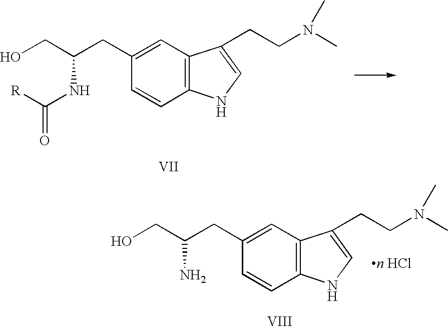
wherein R is same as described above and n=0, 1, 2.

7. The enantioselective process of claim 6, further comprising the step of forming the oxzalidinone ring compound of Formula I by reacting the compound of Formula VIII with a ring closure agent: 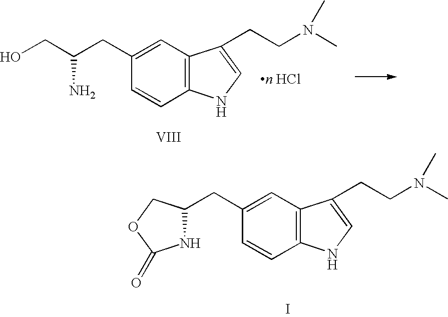
wherein n=0, 1, 2.

8. The enantioselective process of claim 7, wherein said ring closure agent is selected from the group consisting of phosgene, diphosgene, triphosgene, ethyl carbonate and 1,1′ carbonyldiimidazole.

9. The enantioselective process of claim 1, wherein said substituted alanine compound of Formula V is prepared by reacting a compound having the structure of Formula IV: 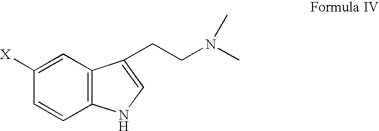
wherein X represents a halogen, with methyl 2-acetamido-acrylate via a Heck Reaction.

10. The enantioselective process of claim 9, wherein said compound of formula IV is prepared by reacting a compound having the structure of Formula III: 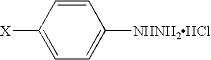
wherein X represents a halogen, with 4-N,N-dimethylbutanal diacetal via a Fischer reaction.

11. The enantioselective process of claim 10, wherein said compound of Formula III is prepared by reacting a 4-halo-aniline with sodium nitrite.

12. A compound having the structural Formula (V): 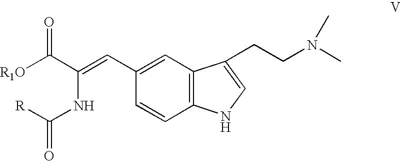
wherein R and R₁ are independently an alkyl group.

13. A compound having the structural Formula (VI): 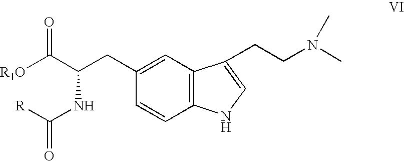
wherein R and R₁ are wherein R and R₁ are independently an alkyl group.

14. A compound having the structural Formula (VI-A): 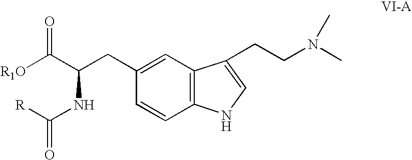
wherein R and R₁ are the same wherein R and R₁ are independently an alkyl group.

15. A compound having the structure of Formula (VII): 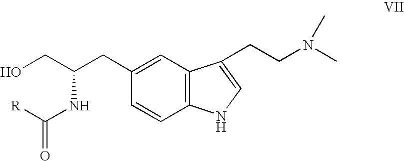
wherein R is an alkyl group.

16. A compound having structural formula (VI-A): 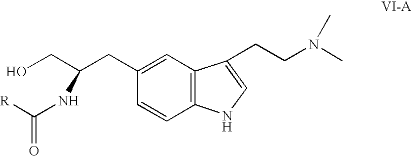
wherein R is an alkyl group.

17. An enantioselective process for producing a substituted (R)-alanine compound having the structure: 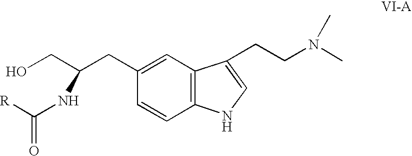
wherein R is an alkyl group, said process comprising asymmetrically hydrogenating a compound having the structure: 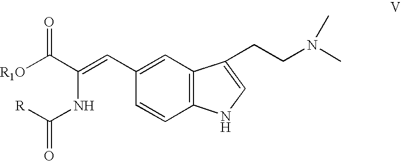
in the presence of hydrogen and an (R)-directing chiral phosphine transition metal catalyst.

18. The process of claim 17, wherein said (R)-directing chiral phosphine transition metal catalyst is an (R)-directing enantioselective P-chiral catalyst represented by the Formula M(L)(P*)X, wherein M represents Rh, Ru or iridium; L represents 1,5-cyclooctandiene or 2,5-norbornadiene; P* represents an (R)-directing chiral monodentate or bidentate phosphine compound, and X represents a tetrahaloborate.

19. The process of claim 17, wherein X is tetrafluoroborate.
